(19) United States
(12) Patent Application Publication
(10) Pub. No.: US 2021/0241874 A1
(43) Pub. Date: Aug. 5, 2021

(54) OVERDOSE DIAGNOSTIC AND TREATMENT DEVICE AND METHOD

(71) Applicant: Definitive Biotechnologies, LLC, Elkridge, MD (US)

(72) Inventors: Timothy Alcorn, Raleigh, NC (US); Pamela Duchars, Steventon Abingdon (GB); Peter Latham, Acton, MA (US)

(21) Appl. No.: 17/163,305

(22) Filed: Jan. 29, 2021

Related U.S. Application Data
(60) Provisional application No. 62/968,276, filed on Jan. 31, 2020.

(51) Int. Cl. G16H 20/10; G16H 10/40; G01N 33/49

(52) U.S. Cl. CPC G16H 20/10; A61B 5/15; G01N 33/4925; G16H 10/40

(57) ABSTRACT

Devices and methods for diagnosing and administering drug overdose countermeasure. In some embodiments, a device includes a analyzer with a decision support application electronically connected to a drug of abuse detection device and a non-invasive blood gas device that support specific identification of drugs of abuse within a patient's system. In some embodiments, a method includes inputting patient demographics into the analyzer, inputting a patient's saliva, blood, and/or urine sample into the drug of abuse detection device, operatively affixing a non-invasive blood gas device to a patient, and administering a proper countermeasure dosage analyzer to the patient experiencing drug overdose.

### RELATED APPLICATIONS

This application claims benefit under 35 U.S.C. § 119(e) to U.S. Provisional Application Ser. No. 62/968,276, filed on Jan. 31, 2020, the entirety of which is hereby incorporated by reference.

### FIELD OF THE DISCLOSURE

The present disclosure generally relates to methods and devices for diagnosing and treating drug overdoses.

### BACKGROUND INFORMATION

Drug addiction is a global epidemic and is viewed as a major health concern in many countries, including the United States. Opioid addiction is particularly dangerous and has been increasing in the United States for a number of years. Contributing to opioid addiction are illicit drugs such as heroin, prescription drugs such as oxycodone, and a variety of synthetic opioids such as fentanyl.

In the United States alone, more than 70,000 people died in 2017 from drug overdoses. Of those deaths, nearly 50,000 were attributable to opioid-specific overdose. Further, abuse of prescription opioids costs the healthcare system about $78.5 billion annually.

Opioids work by chemically interacting with opioid receptors in the brain and nervous system. Generally, opioids are prescribed to relieve pain, but frequently are abused for their euphoric effects. Sufferers of opioid addiction frequently turn to illicitly made fentanyl, which can be 50-100 times more potent than morphine. These illegally-made drugs often contain other drugs, unknown to the user, such as heroin or cocaine. Alternatively, a drug user may unwittingly subject themselves to opioids or synthetic opioids if another illicitly-produced drug has been laced with fentanyl or other opioids.

One of the major symptoms of opioid overdose is Opioid Induced Respiratory Depression (OIRD), which results in decreased blood oxygen concentration and a corresponding spike in exhaled carbon dioxide, causing difficulty in breathing. Other symptoms a patient may exhibit while experiencing an opioid—or other drug—overdose include increased heart rate (tachycardia), confusion, nausea, dizziness, unresponsiveness or loss of consciousness, and pain.

The most common countermeasure for opioid overdose is naloxone and it is the only approved countermeasure currently available. Naloxone is an opioid antagonist, which binds to the opioid receptors in the brain and nervous system, blocking and reversing the effects of opioids. It is widely distributed to healthcare professionals, first responders, patients for in-home use, and it is often made accessible to drug addicts. Currently, naloxone may be administered as a nasal spray, auto-injector, or injection. When properly administered, naloxone is effective at reversing OIRD.

However, with the proliferation of a variety of opioid derivatives, including potent compounds such as fentanyl derivatives, the administration of countermeasures, such as naloxone, is often complex and ineffective. Although naloxone is effective at reversing OIRD when properly administered, it has a variable and limited duration of effect from twenty to ninety minutes depending on multiple factors. This duration is shorter than the duration of the effect of most opioid toxicity, including the effects of some potent fentanyl derivatives. Therefore, naloxone may need to be administered multiple times to counter an opioid overdose. The administration of naloxone can be further complicated by the presence of other respiratory depressants such as barbiturates and alcohol, which are not affected by naloxone.

### SUMMARY

It is therefore an object to provide tools for overdose countermeasure administration that considers the identity and concentration of opioids and other factors, such as patient demographics, that could affect drug metabolism.

The present disclosure is directed, in at least some aspects, to an overdose diagnostic and treatment device and related methods for the diagnosis and treatment of opioid or other drug overdose. It should be understood by those of ordinary skill in the art that the devices and methods can be used for diagnosing and treating overdoses and related overdose symptoms of any drug, such as, but not limited to, morphine, heroin, oxycodone, fentanyl, cocaine, amphetamine, tetrahydrocannabinol (THC), and all related drug classes. An advantage of such devices and methods is that it can be used by first responders, emergency room personnel, physicians, and other healthcare or law enforcement professionals, to guide the administration of countermeasures to a patient experiencing overdose. Additionally, the devices and methods can be utilized in home to assist in countering the symptoms of opioid overdose. Further, the use of the diagnostic and treatment device and method allows for a more accurate and effective administration of overdose countermeasures, such as naloxone, to the patient.

“Patient” refers to a person who is experiencing the overdose and is thus may be treated with overdose countermeasure(s). “User” refers to a person, such as (but not limited to) a medical professional, first responder, or community member and/or family member, who may use the diagnostic and treatment devices and methods on a patient.

In one aspect, a system for detection and countermeasure of drug overdose includes an analyzer and a drug detection apparatus adapted to receive a sample from a person containing at least one drug, determine an identity of the at least one drug, determine a concentration of the at least one drug in the sample, and transmit said identity and concentration to the analyzer. The analyzer is adapted to receive the identity and concentration from the drug detection apparatus, and to receive information about the person including age, ethnicity, sex, weight, and/or race. The analyze is also adapted to determine, based on said identity, concentration, and/or information, an overdose countermeasure for at least partially counteracting an overdose condition of the person. In at least some embodiments, the system includes a blood gas apparatus adapted to measure at least one vital sign of the person including an amount of at least one blood gas in the person's blood and transmit said at least one vital sign to the analyzer. The analyzer is adapted to receive the at least one vital sign from the blood gas apparatus, and to determine, based on the identity, concentration, at least one vital sign and/or information, an overdose countermeasure for at least partially counteracting an overdose condition of the person.

In another aspect, a system for detection and countermeasure of drug overdose includes first means and second means for receiving a sample from a person containing at least one drug. The second means if further for determining an identity of the at least one drug, for determining a concentration of the at least one drug in the sample, and for transmitting said identity and concentration to the first means. The first means is for receiving the identity and concentration from the second means, for receiving information about the person including age, ethnicity, sex, weight, and/or race, and for determining, based on said identity, concentration, and/or information, an overdose countermeasure for at least partially counteracting an overdose condition of the person. Some embodiments include third means for measuring at least one vital sign of the person including an amount of at least one blood gas in the person's blood and for transmitting said at least one vital sign to the first means, and the first means is further for receiving the at least one vital sign from the third means, and for determining, based on the identity, concentration, at least one vital sign and/or information, an overdose countermeasure for at least partially counteracting an overdose condition of the person. In some such embodiments, the first means includes an analyzer, the second means includes a drug detection apparatus, and the third means includes a blood gas apparatus.

In another aspect, a method for treating a drug overdose includes collecting a sample from a person containing at least one drug, inputting the sample into a drug detection apparatus adapted to receive the sample, determine an identity of the at least one drug in the sample, and determine a concentration of the at least one drug in the sample, and inputting into an analyzer information about the person including age, ethnicity, sex, weight, and/or race. The analyzer is operatively connected to the drug detection apparatus and adapted to receive the identity and concentration therefrom. The analyzer is also adapted to determine, based on the identity, concentration, and/or information, an overdose countermeasure for at least partially counteracting an overdose condition of the person. The method further includes perceiving at least one communication from the analyzer specifying a countermeasure for the overdose condition, and administering the countermeasure to the person.

In some embodiments, the method includes operatively connecting a blood gas apparatus to the person, which is adapted to measure at least one vital sign of the person including an amount of at least one blood gas in the person's blood. In some such embodiments, the analyzer is operatively connected to the blood gas apparatus and adapted to receive the at least one vital sign therefrom, and is adapted to determine, based on the identity, concentration, at least one vital sign and/or information, an overdose countermeasure for at least partially counteracting an overdose condition of the person.

In yet another aspect, a method for countermeasure of drug overdose includes receiving an identity and a concentration of at least one drug present in a sample from a person, receiving information about the person including age, ethnicity, sex, weight, and/or race, and determining an overdose countermeasure for at least partially counteracting an overdose condition of the person based on the identity, concentration, and/or information. In further embodiments, the method includes receiving at least one vital sign of the person including an amount of at least one blood gas in the person's blood, and determining an overdose countermeasure for at least partially counteracting an overdose condition of the person based on the identity, concentration, at least one vital sign and/or information.

An additional aspects comprises a software program or non-transitory computer-readable medium having computer-readable instructions stored thereon. When executed by a computer system, the computer system receives an identity and a concentration of at least one drug present in a sample from a person, receives information about the person including age, ethnicity, sex, weight, and/or race, determines an overdose countermeasure for at least partially counteracting an overdose condition of the person based on the identity, concentration, and/or information. In some aspects, the computer system, when executing the program or instructions, receives at least one vital sign of the person including an amount of at least one blood gas in the person's blood, and determines an overdose countermeasure for at least partially counteracting an overdose condition of the person based on the identity, concentration, at least one vital sign and/or information.

In at least some aspects, the overdose diagnostic and treatment device includes a housing case, a power source, a charging port, a sample collection kit, a drug of abuse (DOA) detection apparatus, a non-invasive blood gas apparatus for measuring and monitoring blood gases, and a analyzer with or operatively connected/connected to a decision support application for determining the dosage concentration/amount and timing/frequency of administrations of overdose countermeasure. In at least some embodiments, the housing case contains the power source, charging port, sample collection kit, DOA detection device, non-invasive blood gas device, and analyzer therein. In at least some embodiments, the overdose diagnostic and treatment device further includes an amount of drug overdose countermeasure. Yet further, in some such embodiments, the housing case also includes the amount of drug overdose countermeasure therein.

At least some embodiments are devices and methods for prescribing appropriate and effective concentration of countermeasure to a patient experiencing a drug overdose. At least some embodiments include identification of a patient experiencing a potential drug overdose, collecting a sample of saliva, blood, and/or urine from said patient, inputting the collected sample into a DOA detection device, affixing or operatively connected the non-invasive blood gas device to the patient, inputting information about said patient into the analyzer containing the decision support application, generating instructions or information regarding the administration of a countermeasure or countermeasures, and administering countermeasure(s) to the patient.

In at least some embodiments, the diagnostic and treatment device includes a power source. The power source may include a rechargeable battery and power connection(s) thereto to provide power to components of the diagnostic and treatment device that require power. In at least some such embodiments, the power source is connected and provides power to the DOA detection device, the analyzer, and the non-invasive blood gas device. In at least some embodiments, the power source is housed within the housing case. In some such embodiments, the power source is configured to be charged while the housing case is open or closed via a charging port located within the housing case but externally accessible when the case is closed. In at least some embodiments, the power source is turned on when the housing case is opened, causing the components attached thereto via the power connection to turn on in kind.

In at least some embodiments, the diagnostic and treatment device includes a sample collection kit. In at least some such embodiments, the sample collection kit includes at least one sample collection tool, e.g., a swab for the collection of saliva, a lancet and capillary tube for the collection of blood (e.g., peripheral), and a sample collection cup for a urine sample. Additionally, in at least some such embodiments, the sample collection kit also includes dilution tubes and sample diluent.

In at least some embodiments, the diagnostic and treatment device includes a DOA detection device. In at least some such embodiments, the DOA detection device is a point-of-care device that utilizes high performance electrophoresis in a microfluidic capillary to separate drugs of abuse and a direct optical detection method for detecting and quantitating the drugs of abuse present in the patient's saliva, blood, and/or urine. In at least some such embodiments, the DOA detection device includes a cover, an internal power supply, a waste container, a port for receiving a sample of the patient's, a light source, a photodiode array, a capillary, a mechanism to apply charge across the capillary, a mechanism to generate pressure on the capillary, a wash mechanism, a mechanism to add buffer to the sample, a system control mechanism, a mechanism to acquire and analyze data from the detector, a reference database, and a connectivity port. The sample is injected into the receptacle wherein it is forced or otherwise through a capillary by pressure, charge, solvent liquid flow, and/or capillary action. The DOA detection device detects the types and concentrations of any DOA present in the patient's sample. In at least some embodiments, the communication port allows electronic communication between the DOA detection device and the analyzer via Wi-Fi, USB cable, Bluetooth, and/or other transmission path. In at least some embodiments, the DOA detection device electronically transmits the information regarding the DOA in the patient's sample to the analyzer.

It should be understood by those of ordinary skill in the art that the DOA detection device is not limited to a capillary electrophoretic device. It should be further understood that the DOA detection device is not limited to detecting and quantitating drugs in the patient's saliva, blood, or urine, and other samples from the patient and detection techniques may be used.

In at least some embodiments, the diagnostic and treatment devices and methods include a non-invasive blood gas device, configured to measure and monitor blood gases and respiration transcutaneously. In at least some such embodiments, the non-invasive blood gas device includes a wearable patch and/or cuff, which is placed directly onto the patient, e.g., against or adjacent the skin, and a sensor and control, which includes a communication port, a power supply, a data acquisition mechanism, a gas inlet and outlet, as well as a connection cable to the wearable patch and/or cuff and a gas exchange tube in connection with the wearable patch and/or cuff. In some embodiments, the wearable patch is disposable. In other embodiments, it is reusable. In at least some embodiments, the cuff is a sleeve that is slipped or placed over a patient's appendage. In some embodiments, the user places the patch upon the patient's skin and places the cuff over the patch. In at least some embodiments, the non-invasive blood gas device is configured to measure blood pressure. In at least some embodiments, the non-invasive blood gas device detects and/or measures for respiration, generating information relating to diagnosis and monitoring of patients experiencing OIRD. In at least some embodiments, the communication port allows communication between the non-invasive blood gas device and the analyzer via Wi-Fi, USB cable, Bluetooth, and/or other transmission path.

In at least some embodiments, the diagnostic and treatment device and related method includes an analyzer. In at least some embodiments, the analyzer is a commercially available tablet, or other similarly-equipped technology or computerized device, with at least some embodiments including a rechargeable power source, such as a rechargeable battery. In at least some embodiments, the analyzer has multiple communication modalities, including, but not limited to, cell phone connectivity, Wi-Fi, and Bluetooth connection capabilities. In at least some embodiments, the analyzer is adapted to electronically communicate with the non-invasive blood gas device, wherein the non-invasive blood gas device transmits to the analyzer blood gas concentration(s), respiration information, and/or heart rate information measured from the patient. Additionally, in at least some embodiments, the DOA detection device is in electronic communication with the analyzer, wherein the DOA detection device transmits the results of the DOA detection to the analyzer. The non-invasive blood gas device and/or DOA detection device may be in electronic communication with the analyzer through, but not limited to, Wi-Fi, USB cable connection, and/or Bluetooth connection.

In at least some embodiments, the analyzer is equipped with a decision support application configured to determine dosage concentration/amount and frequency/timing of administrations of a drug overdose countermeasure. In at least some such embodiments, the decision support application utilizes pharmacological data of DOA(s) or DOA class(es), the identity and concentration of each DOA identified in a sample taken from a patient, patient-specific information and/or demographics, such as, but not limited to, age, sex, weight, and race, and vital sign information, such as, but not limited to, heart rate, respiration rate, and blood gases, to guide the overdose countermeasure administration. The decision support application is configured to utilize some or all of said data to generate a recommendation for specific countermeasure(s) to administer to a patient, the dose/amount of said countermeasure(s), the number of doses, and a countdown timer between each administration of the required countermeasure dosage(s). In at least some embodiments, the decision support application is configured to determine whether successful reversal of an overdose condition has been achieved using information regarding the patient's vital signs, e.g., a non-invasive blood gas device. In at least some embodiments, the decision support application receives the data regarding the identity and concentration of each DOA from the DOA detection device. Further, in at least some embodiments, the decision support application receives data regarding heart rate, respiration, and blood gases from the non-invasive blood gas device. Yet further, the decision support application receives the data regarding patient-specific information and/or demographics from user input into the analyzer.

In at least some embodiments, the analyzer includes a graphical user interface, in which instructions, recommendation and information can be displayed to the user regarding how to operate the diagnostic and treatment device and treat the patient. In at least some such embodiments, the graphical user interface contains an input functionality wherein the user may input relevant data regarding the patient, such as, but not limited to, the sex, weight, age, and ethnicity of the patient. In at least some such embodiments, the analyzer receives said inputs and, using the decision support application, determines therefrom the appropriate dosage of countermeasure to administer to the patient.

In at least some embodiments, the analyzer has a human readable touch screen. In at least some embodiments, the analyzer may be equipped with a visible and audible alarm and may issue alerts, such as the patient's countermeasure dosage amount, notifications regarding the next countermeasure dosage and when to administer it, and system maintenance notifications. In at least some such embodiments, the analyzer may display a countdown timer for informing the user when the next dosage of countermeasure should be administered to the patient. In at least some such embodiments, the analyzer displays output data obtained by the non-invasive blood gas device, such as the level of oxygen and/or carbon dioxide in the patient's blood. In at least some such embodiments, the analyzer displays data obtained from the DOA detection device, such as the types of drugs contained within the patient's system and the concentrations of those drugs. Further, in at least some embodiments, the analyzer displays clinical alerts, such as recommending the patient be transported to the hospital.

In at least some embodiments, the analyzer may include therein data storage for certain data including, but not limited to, patient-specific information, data from a DOA detection device, data from a non-invasive blood gas device, analyzer activity logs, information about the decision support application, and/or data regarding drugs of abuse and countermeasure-specific information. Further, in at least some embodiments, the analyzer may include communication protocols for receiving information from a non-invasive blood gas device and/or the DOA detection device, as well as for communicating or receiving information from other external parties or devices.

One advantage of certain embodiments is the speed and accuracy with which the user can diagnose and administer a correct dosage of countermeasure to the patient, as compared to prior systems and processes, especially where the quantities and types of drugs are unknown to the user and the patient is unresponsive or unable to communicate. Where such guesswork is eliminated or reduced, the patient has a higher likelihood of positively responding to the countermeasure. Another advantage of certain embodiments is the ability of non-medically trained users to use the system on patients exhibiting clinical symptoms of a drug overdose.

Those of ordinary skill in the art should understand that, in the future, new countermeasures to drug overdoses may become available. These new countermeasures may have a different mode of action, may be for countering specific drugs or classes of drugs, and may have a different duration of effectiveness. Therefore, these countermeasures may be more appropriate than naloxone in certain situations. This will ultimately lead to an increase in complexity of administering overdose countermeasures, especially where the patient is nonresponsive, or does not know or cannot remember what DOA that patient has taken. Therefore, another advantageous aspect of certain embodiments is that the user will not have to engage in complex or time-consuming medical testing to determine what drugs of abuse are in the patient's system, or what specific countermeasures and in what concentrations will be most effective at treating the patient.

These and other unique features of the device and method disclosed herein will become more readily apparent from the following detailed description of currently preferred embodiments, the accompanying drawings, and the appended claims.

### BRIEF DESCRIPTION OF THE DRAWINGS

The foregoing and other features will be apparent from the following Detailed Description, taken in connection with the accompanying drawings, in which:

FIG. 1 is a schematic view of an overdose diagnostic and treatment device within a housing case;

FIG. 2 is a schematic of an input and decision structure of a device and method for overdose diagnosis and treatment;

FIG. 3 is a schematic view of a DOA detection device of the overdose diagnostic and treatment device of FIG. 1;

FIG. 4 is a schematic view of a non-invasive blood gas device of the overdose diagnostic and treatment device of FIG. 1 for measuring and monitoring blood gases;

FIG. 5 is a schematic of an operation process for an overdose diagnostic and treatment device;

FIG. 6 is a schematic of a continuation of the operation process of FIG. 5;

FIG. 7 is a schematic of a continuation of the operation process of FIG. 6;

FIG. 8 is schematic top perspective view of a non-invasive blood gas device for measuring and monitoring blood gases operatively connected to a analyzer;

FIG. 9 is a schematic view of a housing case and components contained therein; and

FIG. 10 is a schematic view of a graphical user interface of an analyzer and various screenshots thereof.

### DETAILED DESCRIPTION

Described herein are devices and methods for diagnosing and administering countermeasure for the treatment of drug overdose. The devices and methods may be used, for example, for treating patients who are experiencing an overdose of opioids with naloxone. It should be understood, though, that the devices and methods discussed herein may be utilized for many applications, such as treating patients who are experiencing overdose of other drugs or substances, either illicit, prescribed or non-prescription/over-the-counter, with countermeasures, other than naloxone, that may be known or become known to those having ordinary skill in the art.

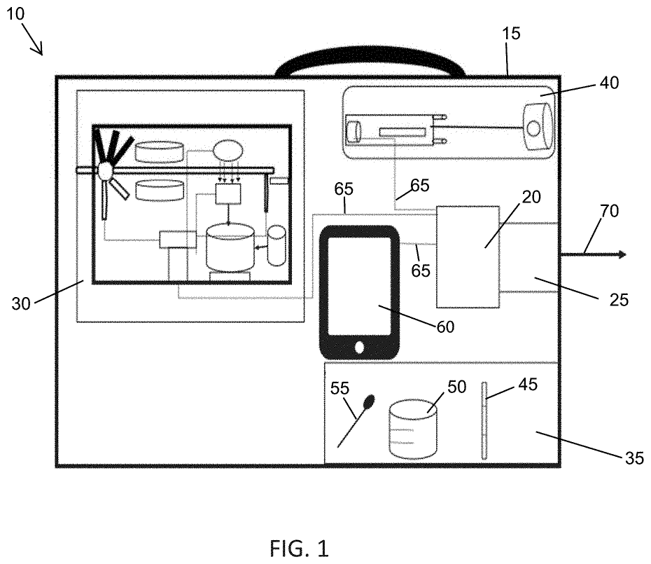

Referring to FIG. 1, a device or system is schematically shown and indicated generally by the reference numeral 10. The device 10 includes a housing case 15 configured to contain the components of the device. Within the housing case 15 are a DOA detection device 30, non-invasive blood gas device 40, power source 20, charging port 25, analyzer 60, and sample collection kit 35. The power source 20 may comprise a rechargeable battery or any other suitable power source as should be understood by those of ordinary skill in the art, including but not limited to one that is also capable of being plugged in or connected to an electrical power line or outlet. The power source 20 is removably attachable to the charging port 25. The charging port 25 is configured to be accessible from the exterior of the housing case 15, as illustrated by line 70, to allow charging of the power source 20 without opening the housing case 15. The charging port 25 is in electrical communication with the power source 20. The power source 20 is electrically/electronically connected, via USB cable, wire, or other similar electrical/electronic connections, with the analyzer 60, the DOA detection device 30, and the non-invasive blood gas device 40, as schematically indicated by lines 65. The sample collection kit 35 contains therein a swab 55, urine sample cup 50, and blood collection capillary 45. As those of ordinary skill in the art should understand, though, the sample collection kit 35 may contain, alternatively or additionally, other suitable sample collection devices that are currently known or later known.

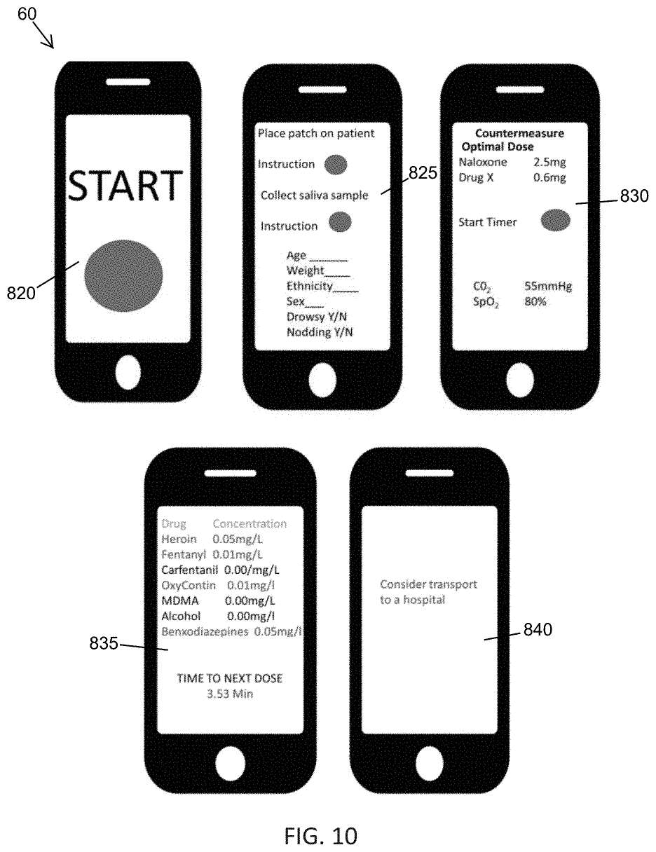

FIG. 9 schematically illustrates one embodiment of components of an opioid overdose diagnostic and treatment device kit. As illustrated, the kit contains a DOA detection device 30, a non-invasive blood gas device 40, a rechargeable battery 805, a sample collection kit 35, a supply of countermeasure 810, an analyzer 60, and a housing case 15.

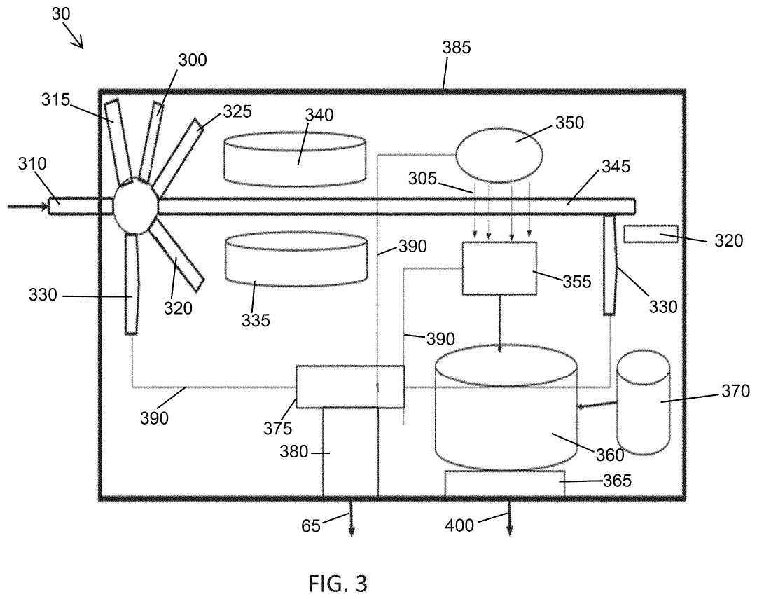

The DOA detection device 30, as illustrated schematically in FIG. 3, includes system control 335, which allows the user to turn on and activate the DOA detection device 30. The DOA detection device 30 further includes a sample receiving port 310 that is in fluid communication with a capillary 345. The patient's sample is introduced into the sample receiving port 310, which is accessible outside of the device housing case 385, and flowed through the capillary 345 using pressure generator 315 and/or charge applicators 330. The charge applicators 330 are used to create a differential voltage (ve+/ve−) from the beginning to the end of the capillary 345. This voltage differential facilitates migration and separation of the DOAs as they enter and exit the capillary 345. A thermal control 340 within the DOA detection device 30 maintains the capillary 345 at a temperature for optimal suitable for separation of different DOAs as they pass through the capillary 345. The DOA detection device 30 also includes a buffer applicator 325 in or placeable in fluid communication with the capillary 345 and the sample receiving port 310, for applying a solvent into the capillary 345 and the sample receiving port 310 for allowing for a solvent, e.g., a liquid, flow with the sample through the capillary 345. The solvent is used for separation of the DOAs as they pass through the capillary 345 by of utilizing the differential solubility of each DOA in a solvent/sample mixture. As the sample passes through the capillary 345, the light source 350, powered by internal power supply 375, which may be charged or energized via power connector 380 which electrically connects to the power source 20 (illustrated via line 65), illuminates so as to pass a stream of light 305 through the capillary 345 containing the sample and onto a multi-pixel photodiode array 355. Each pixel of the array 355 is used to measure light absorption. Light absorption is the mechanism used in this embodiment to detect a DOA as it passes through the capillary 345, as different DOAs have different light absorption characteristics. Measurements from each pixel are combined into a single analysis to increase the signal to noise ratio, thereby increasing the sensitivity of the detection of each DOA. The light source 350 can be capable of generating any specific wavelength of light corresponding to the visible and UV spectra. The photodiode array 355 measures the light absorption as the stream of light 305 passes through the sample and transmits this data to a data acquisition and analysis mechanism 360 contained within the DOA detection device 30. The data acquisition and analysis mechanism 360 measures the time from sample entry into the capillary 325 until the detection of the sample by the photodiode array 355, otherwise known as the “retention time,” and the amount of light absorbed by the target compound being detected. This data is then compared with information regarding retention times and light absorption for drugs of abuse or classes of drugs of abuse contained within an internal reference database 370. This comparison allows the data acquisition and analysis mechanism 360 to determine the identity(ies)/drug class(es) and/or concentration(s) of any DOA contained within the sample. This data is then transmitted through connectivity port 365 to the analyzer 60, schematically illustrated by arrow 400. The transmission may occur via any suitable means known to those of pertinent skill in the art or later developed, including, without limitation, Wi-Fi connection, Bluetooth, USB cable, or the like. The remaining sample exits the capillary 345 and is collected in waste container(s) 320, which can be removed and disposed of (in a safe manner for medical waste). The DOA detection device 30 also includes a wash mechanism 300 whereby any residual sample in the capillary 345 may be cleaned prior to or subsequently after using the DOA detection device 30, e.g., by introducing a cleaner into the capillary 345, and in at least some embodiments, subsequently removing the cleaner from the capillary 345.

The DOA detection device 30 and its methods of operation and/or use may be in accordance with the disclosures and/or teachings of one or more of the following patents, which are hereby incorporated by reference in their entireties as part of the present disclosure: U.S. Pat. No. 7,041,986, entitled “Device for Discrimination of Fluorescence Lifetimes and Uses Therefor”; U.S. Pat. No. 7,718,353, entitled “Proteins, Sensors, and Methods of Characterizing Analytes Using the Same”; and U.S. Pat. No. 8,993,972, entitled “Fluorescence Based Sensors Utilizing a Mirrored Cavity.”

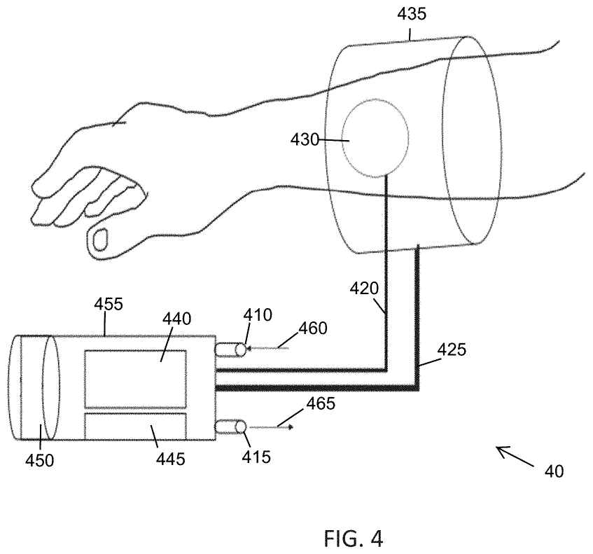

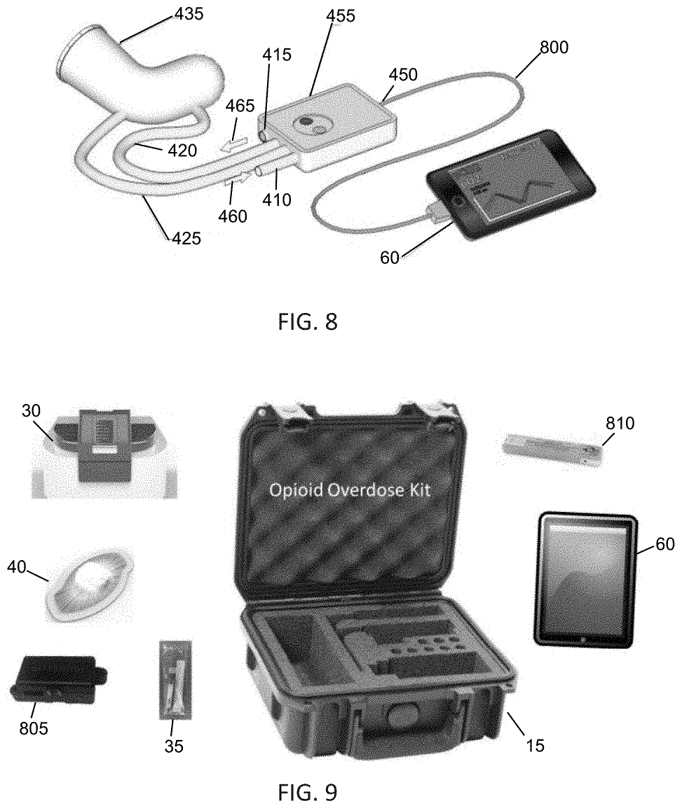

FIG. 4 schematically shows a non-invasive blood gas device 40. The non-invasive blood gas device 40 includes a sensor 455, a wearable patch 430, and gas equilibration cuff 435. The sensor 455 is operatively connected to the wearable patch 430 via connection 420 (e.g., a cable) and the gas equilibration cuff 435 is operatively connected to the sensor 455 via gas exchange tube 425. The sensor 455 includes a gas inlet 410 and a gas outlet 415, wherein gas flow into the sensor 455 is illustrated by arrow 460 and gas flow out of the sensor 455 is illustrated by arrow 465. Housed within the sensor 455 are a data acquisition mechanism 440, a power supply 445, and a communication port 450. The non-invasive blood gas device 40 may contain a commercially available device to measure heart rate/pulse and/or respiration rate in addition to the wearable patch 430. The wearable patch 430 transcutaneously measures blood oxygen (pO₂) and/or blood carbon dioxide (pCO₂). The data obtained by the wearable patch 430 regarding the blood gas(es) is transmitted from the wearable patch 430 and the gas equilibration cuff 435 to the data acquisition mechanism 440 within the sensor 455 via connection 420. The sensor 455 transmits this data through the communication port 450 to the analyzer 60. Referring to FIG. 8, this data may be transmitted via USB cable 800. The data may also be transmitted via Wi-Fi, Bluetooth, other wireless technologies, or any other suitable connection, either currently known or later developed.

The non-invasive blood gas device 40 and its methods of operation and/or use may be in accordance with the disclosures and/or teachings of one or more of the following patents, which are hereby incorporated by reference in their entireties as part of the present disclosure: U.S. Pat. No. 8,852,921, entitled “Non-invasive Sensing of Bioprocess Parameters”; U.S. Pat. No. 9,883,823, entitled “System and Method for Determining an In Vivo Concentration of a Small Molecule Substance of Interest in a Noninvasive Manner”; and U.S. Pat. No. 9,538,944, entitled “Non-invasive Analyte Sensing System and Method.”

The analyzer 60 is a commercially available tablet, or other similarly equipped technology, with a rechargeable battery. However, any suitable analyzer may be used. The illustrated analyzer 60 is recharged via power source 20, and is electronically connected or connectable thereto, as schematically indicated by line 65. The analyzer 60 may have one or more of any suitable communication modalities, including, but not limited to, cell phone connectivity, USB connectivity, Wi-Fi, and Bluetooth connection capabilities. The analyzer 60 includes therein data storage configured for storing certain data including, but not limited to, patient-specific information, DOA detection device 30 output data, non-invasive blood gas device 40 output data, analyzer 60 activity logs, information about the decision support application, and data regarding the drugs of abuse and countermeasure-specific information. The analyzer 60 includes communication protocols for receiving output information from the non-invasive blood gas device 40 and/or the DOA detection device 30, as well as for communicating or receiving information from other external parties or devices.

Referring to FIG. 10, the analyzer 60 has a human-readable touch screen (as illustrated by the schematic screen shots 820, 825, 830, 835, and 840). The analyzer 60 includes a graphical user interface, which displays information regarding the system, e.g., information, messages, instructions, etc. The user may initiate use of the system by pressing the “start” button on screen 820. The analyzer 60 on screen 825 displays instructions and provides prompts wherein the user may input relevant data regarding the patient, such as, but not limited to, the sex, weight, age, and ethnicity of the patient. The analyzer 60 is be equipped with a visible and audible alarm and may generate alerts, such as patient's countermeasure dosage amount, notifications regarding the next countermeasure dosage and when to administer it, and system maintenance notifications, as illustrated on screen 830 in FIG. 10. The analyzer 60 displays a countdown timer, such as illustrated on screen 830 and DOA report screen 835, to inform the user when the next dosage of countermeasure should be administered to the patient, as further discussed below. As illustrated, screen 835 lists DOAs and concentrations thereof detected from the patient sample(s).

Though analyzer 60 is illustrated as having a touch screen, it should be understood that the system may, additionally or alternatively, contain other input/output structures. These include a physical keyboard, which could be either integral or separate from but operatively connected/connectable to the analyzer, voice recognition for data input (e.g., via a microphone), gesture recognition (e.g., via a camera or cameras), and audio transmission to the user. With the latter, for example, the analyzer 60 could audibly provide the user instructions and/or prompt for inputs, e.g., patient data and/or patient sample(s), and/or audibly provide treatment recommendations, reminders/warnings regarding next dose administration, or other instructions, recommendations or information.

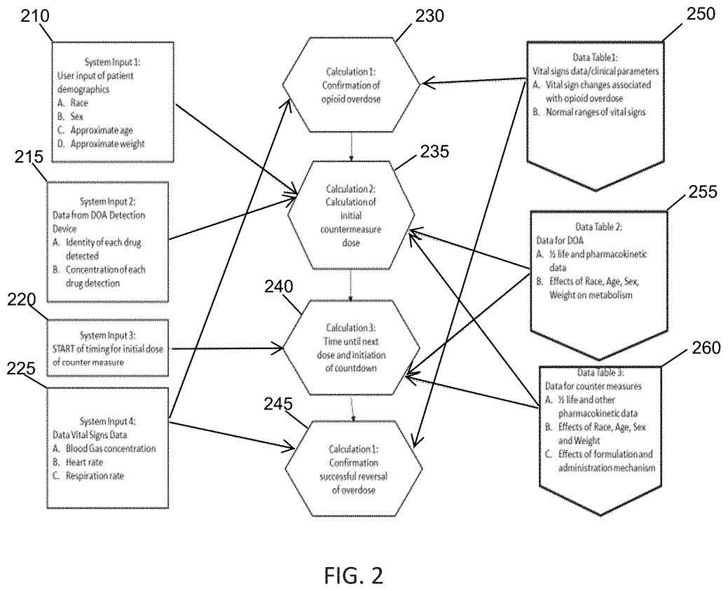

The analyzer 60 supports therein a decision support application. A schematic of an embodiment of a decision support application data input and flow is illustrated in FIG. 2. The decision support application performs determination based on inputs and data table(s). System input 210 includes patient demographics and information, such as, but not limited to, race, sex, age (which may be approximate), and approximate weight (which may be approximate). The user inputs this information into the analyzer 60, for example, through screen 825, as illustrated in FIG. 10, or other method, e.g., as described above. System input 215 includes the data received from the DOA detection device 30, such as the identity of each drug detected (which may in some embodiments be limited to drugs relevant to an overdose condition) and the concentration of each drug. The data included in system input 210 from the DOA detection device 30 may be reported to the user on the DOA report screen 835 on the analyzer 60, as illustrated in FIG. 10. At system input 220, the timer is initiated upon administration of the first dose of countermeasure. For example, a user can initiate this timer by pressing the start timer button on the touchscreen of analyzer 60, such as (but not limited to) the one illustrated on screen 830 in FIG. 10. System input 225 includes the data regarding the patient's vital signs received from the non-invasive blood gas device 40, such as the patient's blood gas concentration(s), heart rate, and/or respiration rate. The analyzer 60 further includes, or is or can be operatively connected to, one or more data tables. Data table 250 includes information regarding vital signs and clinical parameters of drug overdose, such as the vital sign changes associated with overdose and the normal ranges of vital signs. Data table 255 includes information about specific drugs of abuse, such as the half-life and pharmacokinetic information about each drug, as well as the effects of race, age, sex, and weight on a body's ability to metabolize each drug. Data table 260 includes information regarding specific countermeasures, such as the half-life and pharmacokinetic data, the effects of race, age, sex, and weight on a body's ability to metabolize the countermeasures, and the effects of formulation and the administration mechanism of the countermeasures.

It should be understood to those of ordinary skill in the art that, while in some embodiments the decision support application and/or the data tables may be contained within the analyzer, the analyzer may include multiple separate pieces, each containing different components. By way of example only, the data tables and/or decision support application can be housed within separate objects from that which contains the user interface, which are operatively connected or connectable to each other. In yet other embodiments, the data tables and/or decision support application may be located remotely to the analyzer, such as in a separate computer system, server, The Cloud, etc. In such embodiments, the analyzer may communicate with the remote components by any suitable wireless and/or wired communication.

Further referring to FIG. 2, the decision support application uses system inputs 210 through 225 and data tables 250 through 260 to perform determination(s) 230, 235, 240 and 245. The flow of data and information into the various determinations is shown by directional arrows in FIG. 2. Using system input 225 and data table 250, the decision support application performs determination 230, which confirms an overdose condition. Determination 230 compares the patient's vital signs received from system input 225 with the vital signs data and clinical parameters from data table 250 and determines if the patient's vital signs are within a normal range (non-overdose condition) or in a range associated with opioid or other drug overdose. If no overdose is confirmed, the process terminates, and in at least some embodiments generates a message for a user regarding same. On the other hand, once determination 230 has been performed and confirms the overdose, the decision support application proceeds to determination 235, which uses system inputs 210 and 215 and data tables 255 and 260 to perform determination 235. Determination 235 is a determination of the initial countermeasure dosage required for administration to the patient. For each DOA present in the patient's system, and based on its concentration, patient parameters, and pharmacokinetic drug data, the decision support application determines whether the DOA concentration in the patient's system is at a level harmful to the patient. If the DOA concentration is at a level harmful to the patient, the decision support application determines the time (e.g., in minutes) required for the DOA concentration to fall below harmful levels, based on pharmacokinetic data, such as the drug's half-life, and estimated/determined metabolization rates. To determine the countermeasure administration, the drug support application takes into consideration multiple factors, e.g., the specific DOA, the available delivery mechanism of countermeasure to said DOA, patient parameters such as sex, weight, age, and race, and the amount of time for the countermeasure to fall below its effective concentration in the patient (e.g., due to metabolization of same). Following determination 235, the decision support application uses further system input 220 and data tables 255 and 260 to perform determination 240 to determine the amount of time in minutes between the first dosage of countermeasure administered and when the next dosage of countermeasure must be administered to maintain the DOA concentration below harmful levels. If, for example, the effective concentration of the countermeasure is metabolized at a faster rate than the DOA will metabolize to below a harmful concentration, then the decision support application determines the amount of time required between when the first countermeasure dose is administered and when the next dose is required to maintain DOA concentrations below harmful levels, and then for further countermeasure doses, until the DOA concentration in the patient's system falls to a concentration where the drug will no longer cause harm. Thus, the decision support application determines the total number of dosages required and if other measures or interventions must be used. The decision support application's outputs from its determinations regarding dosage, timing, and other interventions are displayed on (or otherwise communicated by) the analyzer 60, such as on the screen 830, the DOA screen 835, and/or screen 840 (which provides a clinical alert), in order to guide the user's administration of countermeasure dosage(s) to the patient. The decision support application performs determination 245 using system input 225 and data table 250 following administration of countermeasure(s) to confirm that the patient's vital signs no longer indicate that the patient is experiencing an overdose condition. Determination 245 compares the patient's vital signs received from system input 225 with the vital signs data and clinical parameters from data table 250 in order to determine if the patient's vital signs are within a normal range or in a range associated with opioid or other drug overdose condition. This confirms the countermeasure is working, or that, after a countermeasure has been metabolized, that intervention is no longer needed, e.g., drug has been sufficiently metabolized.

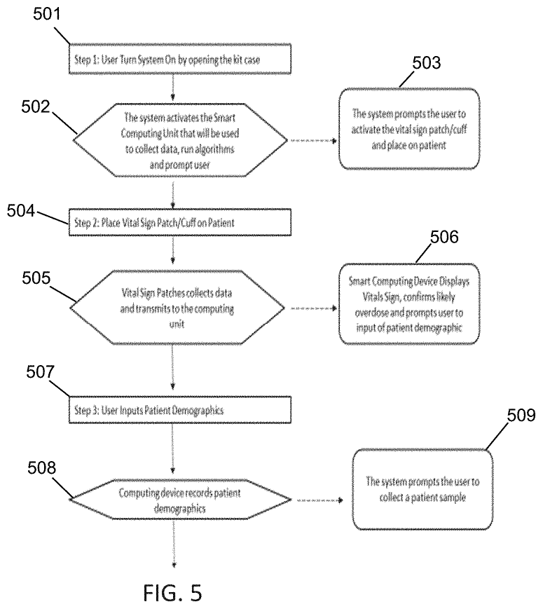

FIGS. 5 through 7 schematically illustrate a method by which the devices described above may be utilized to identify/diagnose and administer countermeasure(s) to a patient.

Referring to FIG. 5, in step 501, the devices inside the housing case 15 are powered on, e.g., simultaneously, upon the user opening the housing case 15 of the overdose diagnostic and treatment device 10. The user may also, in at least some embodiments, turn on (or off) each device individually and/or manually. Upon the analyzer 60 powering on (step 502), the human readable touch screen will display on the graphical user interface instructions the user to activate the non-invasive blood gas device 40 and to place it on or operatively connect it to the patient (step 503). In at least some embodiments, the analyzer 60 will alternatively or additionally provide such instructions by other means, such as audibly. In step 504, the wearable patch 430 and/or gas equilibration cuff 435 of the non-invasive blood gas device 40 are affixed to the patient's appendage, such as an arm, or other suitable body part. In step 505, the non-invasive blood gas device 40 collects data regarding the patient's vitals and transmits it electronically to the analyzer 60. In step 506, the analyzer 60 informs the user if the patient is experiencing an overdose and prompts the user to input the patient information, such as the patient's age, sex, weight, and race. In step 507, the user then enters this data into the analyzer 60. In step 508, the analyzer 60 records this information and, in step 509, prompts the user to collect a sample from the patient using the sample collection kit 35.

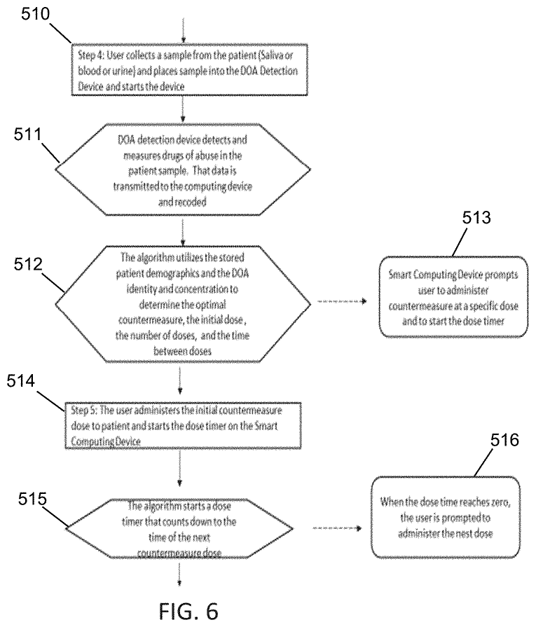

Referring to FIG. 6, in step 510, the user collects a sample of saliva, blood, and/or urine from the patient using the sample collection kit 35 and inserts the sample(s) into the sample receiving port 310 of the DOA detection device 30, starting operation of the DOA detection device 30. In step 511, the DOA detection device 30 detects and measures the concentration of and specific DOA(s) present in the patient's sample(s), and then electronically transmits this information to the analyzer 60, where it is recorded. In step 512, the decision support application utilizes the patient demographics and the DOA identity and concentration to determine a countermeasure, e.g., the initial dosage, the number of doses to be administered, and the time(s) between administrations. In step 513, the analyzer 60 instructs the user to administer a specific dosage of countermeasure(s) and to start the dose timer, for example as illustrated in FIG. 10 on screen 830. In step 514, the user then administers the countermeasure as prescribed by the decision support application and uses the touch screen on the analyzer 60 (or other input) to initiate the decision support application's dose timer. Once the user initiates the timer, in step 515, the decision support application counts down to the time of the next countermeasure dose, for example as illustrated in FIG. 10 on DOA report screen 835. When the timer completely runs out (goes to zero), in step 516, the screen of the analyzer 60 prompts the user to administer the next dosage of countermeasure to the patient (if needed).

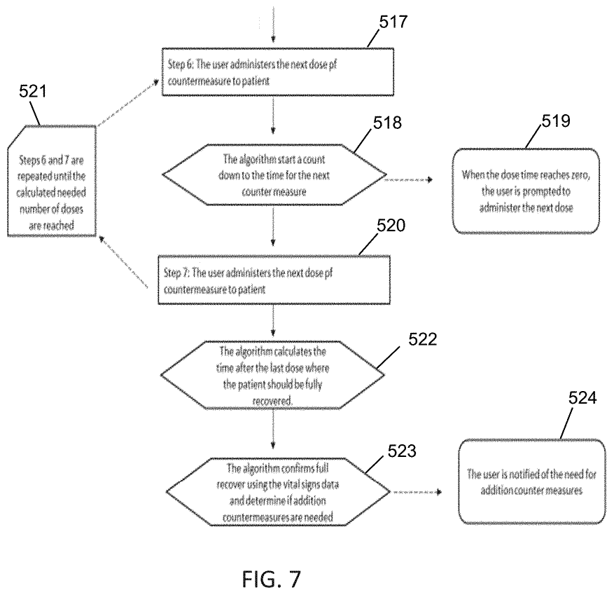

Referring to FIG. 7, in step 517, the user then administers the next dosage of countermeasure as indicated by the decision support application and uses the touch screen on the analyzer 60 (or other input) to initiate the decision support application's timer for that dose. Once the user initiates the timer, in step 518 the decision support application counts down to the time of the next countermeasure dose, for example as illustrated in FIG. 10 on DOA report screen 835. Once the countdown timer has reached zero, the user is prompted to administer the next dosage (step 519) if needed. In step 520, the user administers the next required dose, if there is one, to the patient. At step 521, steps 517 though 520 are repeated until the drug concentration in the patient's body falls to beneath a harmful level, as determined by the decision support application. Once the last dose of countermeasure has been administered, the decision support application in step 522 then calculates the amount of time that it will take after said last countermeasure dose for the patient to be fully recovered. In step 523, the patient's recovery is monitored by the non-invasive blood gas device 40. In at least some embodiments, the non-invasive blood gas device 40 continually or at a suitable interval sends the patient's respiration, blood gas levels, and other vital signs to the analyzer 60. Using this information, the decision support application in determines whether additional countermeasures are necessary, or if any other clinical intervention is required, such as, but not limited to, transporting the patient to the hospital. In step 524, the analyzer 60 will display this information and instruct the user accordingly, for example as illustrated in FIG. 10 on clinical alert screen 840.

As may be recognized by those of ordinary skill in the pertinent art based on the teachings herein, numerous changes, modifications and improvements may be made to the above-described and other embodiments without departing from the spirit of the invention, which is not limited to the appended claims. For example, the sample collection kit may take the form of any of numerous different sample collection kits, that may employ any of numerous different patient sample collecting methods, that are currently known or that later become known. The analyzer may take form of any of numerous different devices, such as tablets, personal computers, laptops, or smartphones, that are currently known or that later become known for performing the respective functions of these devices. Accordingly, this detailed description is to be taken in an illustrative, as opposed to a limiting sense.

1. A system for detection and countermeasure of drug overdose comprising:
an analyzer; and
a drug detection apparatus adapted to receive a sample from a person containing at least one drug, determine an identity of the at least one drug, determine a concentration of the at least one drug in the sample, and transmit said identity and concentration to the analyzer;
wherein the analyzer is adapted to receive said identity and concentration from the drug detection apparatus, and to receive information about the person including age, ethnicity, sex, weight, and/or race, and is further adapted to determine, based on said identity, concentration, and/or information, an overdose countermeasure for at least partially counteracting an overdose condition of the person.

2. A system as defined in claim 1, further including a blood gas apparatus adapted to measure at least one vital sign of the person including an amount of at least one blood gas in the person's blood and transmit said at least one vital sign to the analyzer, wherein the analyzer is adapted to receive the at least one vital sign from the blood gas apparatus, and to determine, based on said identity, concentration, at least one vital sign and/or information, an overdose countermeasure for at least partially counteracting an overdose condition of the person.

3-5. (canceled)

6. A system as defined in claim 2, wherein the analyzer is adapted to determine whether the countermeasure at least partially reverses the overdose condition based on at least one vital sign of the person measured by the blood gas apparatus after administration of the countermeasure to the person.

7. (canceled)

8. A system as defined in claim 1, wherein the countermeasure includes at least one dose of a countermeasure drug.

9. A system as defined in claim 1, wherein the countermeasure includes multiple doses of a countermeasure drug, and the analyzer is further adapted to determine a time period between administration of a first of said multiple doses and a second of said multiple doses.

10-11. (canceled)

12. A system as defined in claim 1, wherein the drug detection apparatus includes a capillary electrophoretic device.

13-16. (canceled)

17. A system as defined in claim 2, wherein the blood gas apparatus is adapted to measure blood oxygen and/or blood carbon dioxide levels of a person.

18-25. (canceled)

26. A system as defined in claim 1, wherein the analyzer includes or is operatively connected or connectable to at least one input device adapted to receive the information about the person from a user of the system.

27. A system as defined in claim 26, wherein the at least one input device includes a touchscreen, a keyboard, a microphone and/or a camera.

28-42. (canceled)

43. A method for treating a drug overdose comprising:
(a) collecting a sample from a person containing at least one drug;
(b) inputting the sample into a drug detection apparatus adapted to receive the sample, determine an identity of the at least one drug in the sample, and determine a concentration of the at least one drug in the sample;
(c) inputting into an analyzer information about the person including age, ethnicity, sex, weight, and/or race, wherein the analyzer is operatively connected to the drug detection apparatus and adapted to receive said identity and concentration therefrom, and determine, based on said identity, concentration, and/or information, an overdose countermeasure for at least partially counteracting an overdose condition of the person;
(d) perceiving at least one communication from the analyzer specifying a countermeasure for the overdose condition; and
(e) administering the countermeasure to the person.

44. A method as defined in claim 43, further including:
(f) operatively connecting a blood gas apparatus to the person, wherein the blood gas apparatus is adapted to measure at least one vital sign of the person including an amount of at least one blood gas in the person's blood;
wherein the analyzer is operatively connected to the blood gas apparatus and adapted to receive the at least one vital sign therefrom and to determine, based on said identity, concentration, at least one vital sign and/or information, an overdose countermeasure for at least partially counteracting an overdose condition of the person.

45-47. (canceled)

48. A method as defined in claim 43, wherein the countermeasure includes multiple doses of the countermeasure drug, and step (e) includes administering to the person a first of said multiple doses at a first time and a second of said multiple doses at a second time later than the first time.

49. A method as defined in claim 48, wherein the at least one communication specifies the second time.

50. A method as defined in claim 48, further including inputting into the analyzer an input substantially representing said first time.

51-52. (canceled)

53. A method as defined in claim 43, including administering the countermeasure until perceiving a further communication from the analyzer specifying to cease administering the countermeasure.

54. A method for countermeasure of drug overdose comprising:
(a) receiving an identity and a concentration of at least one drug present in a sample from a person;
(b) receiving information about the person including age, ethnicity, sex, weight, and/or race; and
(c) determining an overdose countermeasure for at least partially counteracting an overdose condition of the person based on said identity, concentration, and/or information.

55. A method as defined in claim 54, further including:
(d) receiving at least one vital sign of the person including an amount of at least one blood gas in the person's blood;
wherein step (c) comprises determining an overdose countermeasure for at least partially counteracting an overdose condition of the person based on said identity, concentration, at least one vital sign and/or information.

56. (canceled)

57. A method as defined claim 54, wherein step (a) includes receiving said identity and concentration from a drug detection apparatus.

58. A method as defined in claim 55, wherein step (d) includes receiving said at least one vital sign from a blood gas apparatus.

59-62. (canceled)

63. A method as defined in claim 54, further including, after administration of the countermeasure to the person, receiving at least one vital sign of the person and determining whether the countermeasure at least partially reverses the overdose condition based thereon.

64-65. (canceled)

66. A method as defined in claim 54, wherein the countermeasure includes multiple doses of a countermeasure drug, and the method further comprises determining a time period between administration of a first of said multiple doses and a second of said multiple doses.

67-73. (canceled)

74. A non-transitory computer-readable medium having computer-readable instructions stored thereon that, when executed by a computer system, cause the computer system to perform the steps of:
(a) receiving an identity and a concentration of at least one drug present in a sample from a person;
(b) receiving information about the person including age, ethnicity, sex, weight, and/or race; and
(c) determining an overdose countermeasure for at least partially counteracting an overdose condition of the person based on said identity, concentration, and/or information.

75. A computer-readable medium as defined in claim 74, wherein the computer-readable instructions, when executed by a computer system, further cause the computer system to perform the step of
(d) receiving at least one vital sign of the person including an amount of at least one blood gas in the person's blood;
wherein step (c) comprises determining an overdose countermeasure for at least partially counteracting an overdose condition of the person based on said identity, concentration, at least one vital sign and/or information.

76. (canceled)

77. A computer-readable medium as defined in claim 74, wherein step (a) includes receiving said identity and concentration from a drug detection apparatus.

78. A computer-readable medium as defined in claim 75, wherein step (d) includes receiving said at least one vital sign from a blood gas apparatus.

79-82. (canceled)

83. A computer-readable medium as defined in claim 74, wherein the computer-readable instructions, when executed by a computer system, further cause the computer system to perform the steps of, after administration of the countermeasure to the person, determining whether the countermeasure at least partially reverses the overdose condition based on at least one vital sign of the person.

84. (canceled)

85. A computer-readable medium as defined in claim 74, wherein the countermeasure includes multiple doses of a countermeasure drug, and the computer-readable instructions, when executed by a computer system, further cause the computer system to perform the step of determining a time period between administration of a first of said multiple doses and a second of said multiple doses.

86-92. (canceled)
